(19) United States
(12) Patent Application Publication
(10) Pub. No.: US 2022/0373555 A1
(43) Pub. Date: Nov. 24, 2022

(54) Methods to detect or quantify a gadolinium-based contrast agent in a sample using a luminescent agent able to chelate a Gd3+ cation

(71) Applicant: THE REGENTS OF THE UNIVERSITY OF CALIFORNIA, Oakland, CA (US)

(72) Inventors: Roger Molto Pallares, Berkeley, CA (US); Rebecca J. Abergel, Kensington, CA (US)

(73) Assignee: THE REGENTS OF THE UNIVERSITY OF CALIFORNIA, Oakland, CA (US)

(21) Appl. No.: 17/731,313

(22) Filed: Apr. 28, 2022

Related U.S. Application Data
(60) Provisional application No. 63/181,160, filed on Apr. 28, 2021.

(51) Int. Cl. G01N 33/58; G01N 21/76; G01N 21/64

(52) U.S. Cl. CPC G01N 33/582; G01N 21/76; G01N 21/6428

(57) ABSTRACT

The present invention provides for a method to detect and/or quantify a gadolinium-based contrast agents (GBCA) in a sample, the method comprising: (a) providing a sample, (b) contacting the sample with a luminescent agent so that luminescent agent chelates a Gd³⁺ cation of the GBCA, and (c) measuring quenching of a luminescence emission within a range of wavelength; wherein the quenching of the luminescence emission corresponds to the amount of luminescent agent chelating a Gd³⁺ cation.

### CROSS-REFERENCE TO RELATED APPLICATIONS

This application claims priority to U.S. Provisional Patent Application Ser. No. 63/181,160, filed on Apr. 28, 2021, which is hereby incorporated by reference.

### STATEMENT OF GOVERNMENTAL SUPPORT

The invention was made with government support under Contract No. DE-ACO2-05CH11231 awarded by the U.S. Department of Energy. The government has certain rights in the invention.

### FIELD OF THE INVENTION

The present invention is in the field of luminescent agents.

### BACKGROUND OF THE INVENTION

Magnetic resonance imaging (MM) is a diagnostic tool widely used in medicine^{1,2} that has several advantages over other imaging techniques, such as non-invasiveness, sub-millimeter spatial resolution, and lack of radiation burden.³ The technique, however, is limited by its relatively low sensitivity, which requires image contrast enhancement through the addition of magnetic compounds known as MRI contrast agents.⁴ These compounds shorten the relaxation times of water protons,⁵ allowing abnormal tissue to appear brighter than surrounding tissue. The most common contrast agents are comprised of gadolinium cations chelated by linear or macrocyclic ligands⁶ and are employed in approximately 25 to 30% of all MRI procedures.⁷ Gadolinium, however, is not an innocuous element as suggested by several disorders associated with its use.^{7,8} For instance, the administration of gadolinium-based contrast agents (GBCA) to patients with renal dysfunction has been strongly associated with consequential nephrogenic systemic fibrosis,^{9,10} and the accumulation of gadolinium has been observed in the brains of healthy individuals who underwent MRI scan with GBCAs.¹¹⁻¹³ As a result, the U.S. Food and Drug Administration (FDA) issued a public warning against retention of GBCAs in the body, with special attention to contrast agents based on linear ligands.¹⁴ Furthermore, the European Medicines Agency recommended the suspension of the linear contrast agent Magnevist®.¹⁵ For patients who have undergone MRI procedures, rigorous follow up is beneficial to ensure that all administrated GBCAs are excreted through urine and that no adverse effects occur. The gold standard technique for quantifying gadolinium in urine is mass spectrometry, which can be both cost- and time-intensive, and further requires sample digestion before analysis. Although several assays can detect free Gd³⁺ cations in buffer,¹⁶⁻¹⁹ to the best of our knowledge, there is no assay capable of quantifying GBCAs in urine.

Siderophores are a class of biomolecules produced by fungi, bacteria, and some plants that transport iron across cell membranes.²⁰ Bio-inspired siderophore analogues containing multiple metal binding units, such as bidentate catecholamide or hydroxypyridinone groups, linked to polyamine scaffolds show high affinity and selectivity for a wide variety of metal ions.^{21,22} The octadentate 3,4,3-LI(1,2-HOPO) is a chelator that is comprised of four 1-hydroxy-pyridin-2-one (1,2-HOPO) subunits linked to a central spermine scaffold (FIG. 1B) and has been explored for the decorporation off-block elements.²³ Furthermore, 1,2-HOPO groups can sensitize the emission of several lanthanides, including Eu³⁺, which has been explored for bioassay development.²⁴⁻²⁶ Because the affinity of 3,4,3-LI(1,2-HOPO) for lanthanides increases as the 4 f orbitals are gradually filled,²⁷ Gd³⁺ cations in solution could potentially compete with Eu³⁺ and prevent its binding within the Eu³⁺-3,4,3-LI(1,2-HOPO) complex, quenching its emission. Although many bioassays rely on luminescence quenching,^{28,29} displacement of europium by gadolinium cations has never been explored for analytical applications. Moreover, as a synthetic siderophore derivative, 3,4,3-LI(1,2-HOPO) shows higher binding affinity for lanthanides than internal biomolecules or chelators currently used in clinical settings,²³ which suggests that 3,4,3-LI(1,2-HOPO) could acquire Gd³⁺ cations from other coordinating species.

### SUMMARY OF THE INVENTION

The present invention provides for a method to detect and/or quantify a gadolinium-based contrast agents (GBCA) in a sample, the method comprising: (a) providing a sample, (b) contacting the sample with a luminescent agent so that luminescent agent chelates a Gd³⁺ cation of the GBCA, and (c) measuring quenching of a luminescence emission within a range of wavelength; wherein the quenching of the luminescence emission corresponds to the amount of luminescent agent chelating a Gd³⁺ cation.

In some embodiments, the providing step (a) comprises obtaining the sample from a subject. In some embodiments, the sample obtained from the subject is not further treated, prepared, or digested prior to the contacting step (b). In some embodiments, the contacting step (b) comprises incubating the sample and the luminescent agent together. In some embodiments, the incubating lasts for at least 1, 2, 3, 4, 5, 6, 7, 8, 9, or 10 minutes.

In some embodiments, the measuring step (c) comprises: (i) exposing the luminescent agent to one or more, or a range of, wavelengths of light to cause the luminescent agent to produce the luminescence emission, (ii) measuring the amount of luminescence emission, and (iii) comparing the amount of luminescence emission measured in (ii) to an amount of luminescence emission of a luminescent agent mixed with a blank, negative sample control, or not contacted to the sample.

The presence of any GBCA would quench the luminescent agent by replacing the EuC³⁺ with the Gd³⁺ cation of the GBCA. In some embodiments, the chelating agent in contacting step (b) is chelated to Eu³⁺. In the presence of Gd³⁺, the Gd³⁺ cation displaces the Eu' so that chelating agent chelates Gd³⁺.

In some embodiments, the range of wavelength is within the visible spectra. In some embodiments, the constituents of the sample (except for the luminescent agent introduced, added, or mixed with the sample) are not optically active, or not appreciably or significantly optically active, in the range of wavelength. In some embodiments, all of the constituents in the sample contribute less than about 10%, 5%, 1%, 0.5%, or 0.1% of the emission of the luminescent agent introduced, added, or mixed with the sample.

In some embodiments, the range of wavelength is from about 590 nm to 640 nm. In some embodiments, the range of wavelength is from about 580 nm to 630 nm. In some embodiments, the range of wavelength is from about 610 nm to 630 nm. In some embodiments, the range of wavelength is from about 615 nm to 625 nm. In some embodiments, the range of wavelength is about 620 nm.

In some embodiments, the gadolinium-based contrast agent (GBCA) is gadopentetate, gadobenate, or gadodiamide. In some embodiments, the gadopentetate is Magnevist® (gadopentetate dimeglumine; Bayer HealthCare Pharmaceuticals Inc., Whippany, N.J.). In some embodiments, the gadobenate is Multihance® (gadobenate dimeglumine; Bracco Diagnostics Inc., Monroe Township, N.J.). In some embodiments, the gadodiamide is Omniscan® (gadodiamide, gadolinium-based contrast agent; GE Healthcare Inc., Princeton, N.J.).

In some embodiments, the sample is a bodily fluid or a fluid obtained from a body, such as blood, serum, or urine. In some embodiments, the subject is a subject that has been exposed to a GBCA, or had GBCA introduced into the body, gut, or bloodstream of the subject. In some embodiments, the sample is obtained from a subject, such as a human. In some embodiments, the subject is a farm animal, lab animal, or a pet. In some embodiments, the subject is a mammal, such as a horse, cattle, cow, goat, sheep, dog, cat, rabbit, guinea pig, rat, or mouse. In some embodiments, the subject is a bird, such as a chicken, turkey, duck, or goose.

The present invention provides for an assay to detect and quantify contrast agents in urine based on the luminescence quenching of a bioinspired probe, Eu³⁺-3,4,3-LI(1,2-HOPO). Gadolinium-based contrast agents prevent the formation of the Eu³⁺-3,4,3-LI(1,2-HOPO) complex subsequently decreasing the luminescence of the assay solution. Three commercial contrast agents, Magnevist® (gadopentetate dimeglumine; Bayer HealthCare Pharmaceuticals Inc., Whippany, N.J.), Multihance® (gadobenate dimeglumine; Bracco Diagnostics Inc., Monroe Township, N.J.) and Omniscan® (gadodiamide, gadolinium-based contrast agent; GE Healthcare Inc., Princeton, N.J.), are used to demonstrate the analytical concept in synthetic human urine, and subsequent quantification of mouse urine samples. The present invention is the first assay that is capable of detecting and quantifying gadolinium-based contrast agents in urine without sample preparation or digestion. Hydrogen chloride 0.1 N and 6 N from VWR International (Radnor, PA). Surine™ negative urine control, europium (III) chloride hexahydrate 99.99%, gadolinium (III) chloride hexahydrate 99%, 2-(N-morpholino) ethanesulfonic acid (MES), nitric acid (70%) and sodium hydroxide 97% from Sigma-Aldrich (St. Louis, Mo.).

Gadolinium-based contrast agents are widely used in magnetic resonance imaging procedures to enhance image contrast. Despite their ubiquitous use in clinical settings, gadolinium is not an innocuous element, as suggested by several disorders associated with its use. Therefore, novel analytical technologies capable of tracking contrast agent excretion through urine are necessary for optimizing patient safety after imaging procedures.

The invention does not require the use of a mass spectrometry to detect a gadolinium-based contrast agents in a sample, such as urine.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1A. Structures of 5-LIO(Me-3,2-HOPO) (“5LIO”) and 3,4,3-LI(1,2-HOPO) (“343LI”).

FIG. 1B. Structure of linear-spermine-linked 3,4,3-LI(1,2-HOPO). The metal binding oxygen atoms are highlighted in red

FIG. 2. Optical response of the Eu(III)-3,4,3-LI(1,2-HOPO) complex. (a) Absorbance and emission intensity of 20 μM Eu(III)-3,4,3-LI(1,2-HOPO) in a.u. (b) Optical response of 20 μM complex to 50 μM Gd³⁺ and other cations and subsequent (c) emission quenching at 620 nm. All experiments were performed in duplicate and error bars represent one standard deviation of the measurements

FIG. 3. Mechanism of assay response to GBCA. The metal binding oxygen atoms are highlighted in red. The insets are photos of the sensing solutions with and without Gd³⁺ under UV (320 nm) illumination.

FIG. 4. Analytical response of the assay to GBCA in urine. (a) Molecular structures of each GBCA analyzed. Emission spectra of 20 μM bio-inspired complex to different concentrations of (b) Magnevist®, (c) Multihance®, and (d) Omniscan® in 50% urine and 50 mM MES (pH 6.0). Emission spectra were recorded under 315 nm excitation. (e) Assay response curve of the three GBCAs studied, in 50% synthetic human urine and 50 mM MES (pH 6.0). All experiments were performed in triplicate and error bars represent one standard deviation of the measurements.

FIG. 5. Effect of urine in Eu³⁺-3,4,3-LI(1,2-HOPO) emission. Emission spectrum of 20 μM Eu³⁺-3,4,3-LI(1,2-HOPO) in different urine content under excitation at 315 nm.

FIG. 6. Lack of urine emission. Emission spectra of 50% and 100% synthetic human urine samples under 315 nm excitation.

FIG. 7. Calibration curves used for mouse urine sample quantification. Calibration curves for (top) Magnevist®, (center) Multihance®, and (bottom) Omniscan®. The standard deviations of the Omniscan® measurements were too small for the error bars to be visible. Emission quenching at 620 nm under 315 nm excitation.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

It must be noted that as used herein and in the appended claims, the singular forms “a”, “and”, and “the” include plural referents unless the context clearly dictates otherwise. Thus, for example, reference to “a chelating agent” includes a plurality of such chelating agents, and so forth.

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The 1,2-HOPO and 3,2-HOPO chelating agents

In some embodiments, the luminescent agent is a 1,2-HOPO chelating agent or 3,2-HOPO chelating agent. The 1,2-HOPO and 3,2-HOPO chelating agents suitable for use in the present invention are taught in U.S. Pat. No. 4,698,431 (“Hydroxypyridonate Chelating Agents”), U.S. Pat. No. 5,634,901 (“3-Hydroxy-2(1H)-pyridonate Chelating Agents”), and U.S. Pat. No. 5,892,029 (“3-Hydroxy-2(1H)-pyridonate Chelating Agents”), all of which are hereby incorporated by reference.

Suitable 1,2-HOPO chelating agent include, but are not limited to, molecules defined by the structure:

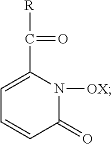

wherein R is a hydroxy group or

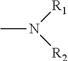

where R₁ and R₂ are selected from the group consisting of H, —CH₃, —CH₂CH₃ and —CH₂-φ, and X is either hydrogen, an alkali metal ion, or a quaternary ammonium ion.

Suitable 1,2-HOPO chelating agent include, but are not limited to, molecules incorporating a plurality of HOPO-type structures, including:

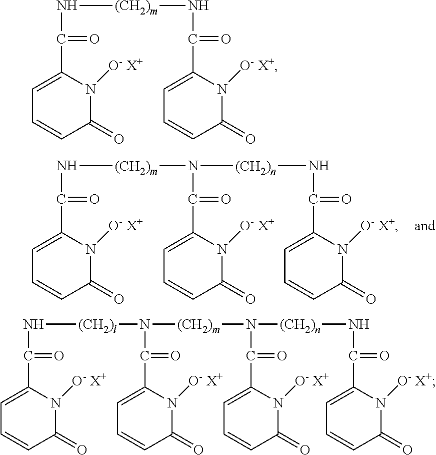

wherein l, m and n are integers between one and twenty. In a particular embodiment of the invention, m is three. In a particular embodiment of the invention, m is three and n is four. In a particular embodiment of the invention, 1 and n are three, and m is four

Suitable 1,2-HOPO and 3,2-HOPO chelating agents include, but are not limited to, a chelating agent comprised of a plurality of chelating functional units joined by one or more linking members, said chelating functional units independently selected from the group consisting of

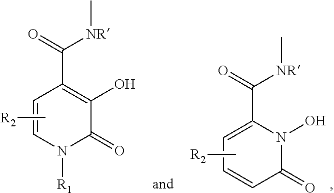

in which at least one of said plurality of chelating functional units on said chelating agent is

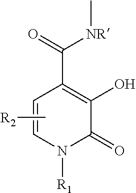

wherein R₁ and R₂ are independently selected from the group consisting of hydrogen, C₁₋₄ aliphatic hydrocarbon groups, and C₁₋₄ aliphatic hydrocarbon groups substituted by a single halide, hydroxy, carboxy, acrylamido group or an aryl group, and R′ is a member selected from the group consisting of a bond to a linking member, a hydrogen atom, C₁₋₈ aliphatic hydrocarbon groups, aryl groups, and C₁₋₈ aliphatic hydrocarbon groups substituted by amino, carboxy, or hydroxy groups.

Suitable 3,2-HOPO chelating agents include, but are not limited to, a chelating agent having the structure:

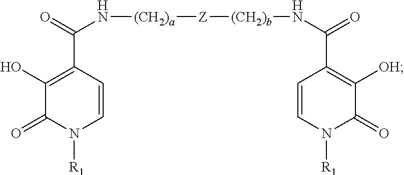

wherein R₁ is a member selected from the group consisting of hydrogen, C₁₋₄ aliphatic hydrocarbon groups, and C₁₋₄ aliphatic hydrocarbon groups substituted by a single halide, hydroxy, carboxy, or aryl group;
Z is a member selected from the group consisting of O, NH, N-alkyl, and N-aryl;
a is 2-4; and
b is 2-4.

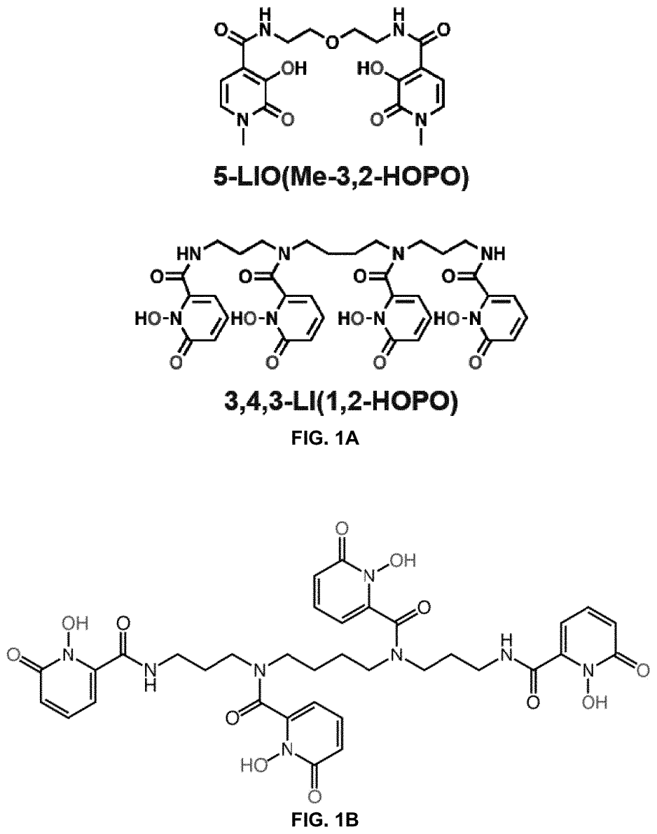

A suitable 1,2-HOPO and a suitable 3,2-HOPO are shown in FIG. 1A.

The methods for synthesizing the 1,2-HOPO and 3,2-HOPO chelating agents are taught in U.S. Pat. Nos. 4,698,431; 5,634,901; and 5,892,029, all of which are hereby incorporated by reference.

References cited herein (which are all individually and specifically incorporated by reference):

(1) Glover, G. H. Overview of functional magnetic resonance imaging. Neurosurg Clin N Am 2011, 22 (2), 133-vii.
(2) Zhi-Pei, L.; Paul, C. L. Introduction. In Principles of Magnetic Resonance Imaging: A Signal Processing Perspective; IEEE: 2000; pp 1-11.
(3) Webb, A.; Kagadis, G. C. Introduction to Biomedical Imaging. Medical Physics 2003, 30 (8), 2267-2267.
(4) Yan, G. P.; Robinson, L.; Hogg, P. Magnetic resonance imaging contrast agents: Overview and perspectives. Radiography 2007, 13, e5-e19.
(5) Tóth, é.; Helm, L.; Merbach, A. Relaxivity of Gadolinium(III) Complexes: Theory and Mechanism. In The Chemistry of Contrast Agents in Medical Magnetic Resonance Imaging; Wiley Hoboken, N.J., 2013; pp 25-81.
(6) Kotek, J.; Kubiček, V.; Hermann, P.; Lukeš, I. Synthesis and Characterization of Ligands and their Gadolinium(III) Complexes. In The Chemistry of Contrast Agents in Medical Magnetic Resonance Imaging; Wiley: Hoboken, N.J., 2013; pp 83-155.
(7) Idée, J. M.; Port, M.; Raynal, I.; Schaefer, M.; Le Greneur, S.; Corot, C. Clinical and biological consequences of transmetallation induced by contrast agents for magnetic resonance imaging: a review. Fundam. Clin. Pharmacol. 2006, 20 (6), 563-576.
(8) Shellock, F. G.; Kanal, E. Safety of magnetic resonance imaging contrast agents. Journal of Magnetic Resonance Imaging 1999, 10 (3), 477-484.
(9) Grobner, T. Gadolinium—a specific trigger for the development of nephrogenic fibrosing dermopathy and nephrogenic systemic fibrosis? Nephrology Dialysis Transplantation 2006, 21 (4), 1104-1108.
(10) Kuo, P. H.; Kanal, E.; Abu-Alfa, A. K.; Cowper, S. E. Gadolinium-based MR Contrast Agents and Nephrogenic Systemic Fibrosis. Radiology 2007, 242 (3), 647-649.
(11) Kanda, T.; Fukusato, T.; Matsuda, M.; Toyoda, K.; Oba, H.; Kotoku, J. i.; Haruyama, T.; Kitajima, K.; Furui, S. Gadolinium-based Contrast Agent Accumulates in the Brain Even in Subjects without Severe Renal Dysfunction: Evaluation of Autopsy Brain Specimens with Inductively Coupled Plasma Mass Spectroscopy. Radiology 2015, 276 (1), 228-232.
(12) Robert, P.; Violas, X.; Grand, S.; Lehericy, S.; Idée, J. M.; Ballet, S.; Corot, C. Linear Gadolinium-Based Contrast Agents Are Associated With Brain Gadolinium Retention in Healthy Rats. Invest Radiol 2016, 51 (2), 73-82.
(13) Kanda, T.; Oba, H.; Toyoda, K.; Kitajima, K.; Furui, S. Brain gadolinium deposition after administration of gadolinium-based contrast agents. Japanese Journal of Radiology 2016, 34 (1), 3-9.
(14) FDA Drug Safety Communication: FDA evaluating the risk of brain deposits with repeated use of gadolinium-based contrast agents for magnetic resonance imaging (MRI). U.S. Food and Drug Administration: 2017.
(15) Dekkers, I. A.; Roos, R.; van der Molen, A. J. Gadolinium retention after administration of contrast agents based on linear chelators and the recommendations of the European Medicines Agency. European Radiology 2018, 28 (4), 1579-1584.
(16) Zare-Dorabei, R.; Norouzi, P.; Ganjali, M. R. Design of a Novel Gadolinium Optical Sensor Based on Immobilization of (Z)-N′-((Pyridine-2-yl) Methylene) Thiophene-2-carbohydrazide on a Triacetylcellulose Membrane and Its Application to the Urine Samples. Anal. Lett. 2009, 42 (1), 190-203.
(17) Zare-Dorabei, R.; Norouzi, P.; Ganjali, M. R. Design of a novel optical sensor for determination of trace gadolinium. J. Hazard. Mater. 2009, 171 (1), 601-605.
(18) Zamani, H. A.; Faridbod, F.; Ganjali, M. R. A new selectophore for gadolinium selective sensor. Materials Science and Engineering: C 2014, 43, 488-493.
(19) Pallares, R. M.; Carter, K. P.; Zeltmann, S. E.; Tratnjek, T.; Minor, A. M.; Abergel, R. J. Selective Lanthanide Sensing with Gold Nanoparticles and Hydroxypyridinone Chelators. Inorg. Chem. 2020, 59 (3), 2030-2036.
(20) Neilands, J. B. Siderophores: Structure and Function of Microbial Iron Transport Compounds. J. Biol. Chem. 1995, 270 (45), 26723-26726.
(21) Deblonde, G. J. P.; Sturzbecher-Hoehne, M.; Abergel, R. J. Solution Thermodynamic Stability of Complexes Formed with the Octadentate Hydroxypyridinonate Ligand 3,4,3-LI(1,2-HOPO): A Critical Feature for Efficient Chelation of Lanthanide(IV) and Actinide(IV) Ions. Inorg. Chem. 2013, 52 (15), 8805-8811.
(22) Sturzbecher-Hoehne, M.; Choi, T. A.; Abergel, R. J. Hydroxypyridinonate Complex Stability of Group (IV) Metals and Tetravalent f-Block Elements: The Key to the Next Generation of Chelating Agents for Radiopharmaceuticals. Inorg. Chem. 2015, 54 (7), 3462-3468.
(23) Rees, J. A.; Deblonde, G. J. P.; An, D. D.; Ansoborlo, C.; Gauny, S. S.; Abergel, R. J. Evaluating the potential of chelation therapy to prevent and treat gadolinium deposition from MM contrast agents. Scientific Reports 2018, 8 (1), 4419.
(24) Abergel, R. J.; D'Aléo, A.; Ng Pak Leung, C.; Shuh, D. K.; Raymond, K. N. Using the Antenna Effect as a Spectroscopic Tool: Photophysics and Solution Thermodynamics of the Model Luminescent Hydroxypyridonate Complex [EuIII(3,4,3-LI(1,2-HOPO))]-. Inorg. Chem.
(25) Daumann, L. J.; Tatum, D. S.; Snyder, B. E. R.; Ni, C.; Law, G. l.; Solomon, E. I.; Raymond, K. N. New Insights into Structure and Luminescence of EuIII and SmIII Complexes of the 3,4,3-LI(1,2-HOPO) Ligand. J. Am. Chem. Soc. 2015, 137 (8), 2816-2819.
(26) Pallares, R. M.; Abergel, R. J. Transforming lanthanide and actinide chemistry with nanoparticles. Nanoscale 2020, 12 (3), 1339-1348.
(27) Sturzbecher-Hoehne, M.; Ng Pak Leung, C.; D'Aléo, A.; Kullgren, B.; Prigent, A. L.; Shuh, D. K.; Raymond, K. N.; Abergel, R. J. 3,4,3-LI(1,2-HOPO): In vitro formation of highly stable lanthanide complexes translates into efficacious in vivoeuropium decorporation. Dalton Trans. 2011, 40 (33), 8340-8346.
(28) Pallares, R. M.; Sutarlie, L.; Thanh, N. T. K.; Su, X. Fluorescence sensing of protein-DNA interactions using conjugated polymers and graphene oxide. Sensors and Actuators B: Chemical 2018, 271, 97-103.
(29) Pallares, R. M.; Thanh, N. T. K.; Su, X. Sensing of Circulating Cancer Biomarkers with Metal Nanoparticles. Nanoscale 2019, 11, 22152-22171.

### (30) Wu, A. H. B.; Bristol, B.; Sexton, K.; Cassella-McLane, G.; Holtman, V.; Hill, D. W. Adulteration of Urine by “Urine Luck”. Clinical Chemistry 1999, 45 (7), 1051-1057.

(31) Chen, R. F. Fluorescence Quantum Yields of Tryptophan and Tyrosine. Anal. Lett. 1967, 1 (1), 35-42.
(32) Telgmann, L.; Holtkamp, M.; Kunnemeyer, J.; Gelhard, C.; Hartmann, M.; Klose, A.; Sperling, M.; Karst, U. Simple and rapid quantification of gadolinium in urine and blood plasma samples by means of total reflection X-ray fluorescence (TXRF). Metallomics 2011, 3 (10), 1035-1040.
(33) Deng, T.; Mai, Z.; Cai, C.; Duan, X.; Zhu, W.; Zhang, T.; Wu, W.; Zeng, G. Influence of weight status on 24-hour urine composition in adults without urolithiasis: A nationwide study based on a Chinese Han population. PLoS One 2017, 12 (9), e0184655.
(34) Shah, R.; LaCourse, W. R. An improved method to detect ethyl glucuronide in urine using reversed-phase liquid chromatography and pulsed electrochemical detection. Anal. Chim. Acta 2006, 576 (2), 239-245.
(35) Mishra, B.; Basu, A.; Chua, R. R. Y.; Saravanan, R.; Tambyah, P. A.; Ho, B.; Chang, M. W.; Leong, S. S. J. Site specific immobilization of a potent antimicrobial peptide onto silicone catheters: evaluation against urinary tract infection pathogens. J. Mater. Chem. B 2014, 2 (12), 1706-1716.
(36) Mullen, J. E.; Thörngren, J. O.; Schulze, J. J.; Ericsson, M.; Gårevik, N.; Lehtihet, M.; Ekstrom, L. Urinary steroid profile in females—the impact of menstrual cycle and emergency contraceptives. Drug Testing and Analysis 2017, 9 (7), 1034-1042.
(37) Maalouf, N. M.; Cameron, M. A.; Moe, O. W.; Adams-Huet, B.; Sakhaee, K. Low Urine pH: A Novel Feature of the Metabolic Syndrome. Clinical Journal of the American Society of Nephrology 2007, 2 (5), 883-888.
(38) Uggeri, F.; Aime, S.; Anelli, P. L.; Botta, M.; Brocchetta, M.; de Haeen, C.; Ermondi, G.; Grandi, M.; Paoli, P. Novel Contrast Agents for Magnetic Resonance Imaging. Synthesis and Characterization of the Ligand BOPTA and Its Ln(III) Complexes (Ln=Gd, La, Lu). X-ray Structure of Disodium (TPS-9-145337286-C-S)-[4-Carboxy-5,8,11-tris(carboxymethyl)-1-phenyl-2-oxa-5,8,11-triazatridecan-13-oato(5-)]gadolinate(2-) in a Mixture with Its Enantiomer. Inorg. Chem. 1995, 34 (3), 633-643.
(39) Loreti, V.; Bettmer, J. Determination of the MM contrast agent Gd-DTPA by SEC-ICP-MS. Anal. Bioanal. Chem. 2004, 379 (7), 1050-1054.
(40) Alwasiyah, D.; Murphy, C.; Jannetto, P.; Hogg, M.; Beuhler, M. C. Urinary Gadolinium Levels After Contrast-Enhanced MRI in Individuals with Normal Renal Function: a Pilot Study. Journal of Medical Toxicology 2019, 15 (2), 121-127.
(41) Lancelot, E. Revisiting the Pharmacokinetic Profiles of Gadolinium-Based Contrast Agents: Differences in Long-Term Biodistribution and Excretion. Invest Radiol 2016, 51 (11), 691-700.
(42) Yang, B.; Bankir, L. Urea and urine concentrating ability: new insights from studies in mice. American Journal of Physiology-Renal Physiology 2005, 288 (5), F881-F896.
(43) Salonia, J. A.; Gasquez, J. A.; Martinez, L. D.; Cerutti, S.; Kaplan, M.; Olsina, R. A. Inductively Coupled Plasma Optical Emission Spectrometric Determination of Gadolinium in Urine Using Flow Injection On-Line Sorption Preconcentration in a Knotted Reactor. Instrum. Sci. Technol. 2006, 34 (3), 305-316.

### Example 1

### Rapid Detection of Gadolinium-Based Contrast Agents in Urine with a Chelated Europium Luminescent Probe

Gadolinium-based contrast agents are widely used in magnetic resonance imaging procedures to enhance image contrast. Despite their ubiquitous use in clinical settings, gadolinium is not an innocuous element, as suggested by several disorders associated with its use. Therefore, novel analytical technologies capable of tracking contrast agent excretion through urine are necessary for optimizing patient safety after imaging procedures. Herein is described an assay to detect and quantify contrast agents in urine based on the luminescence quenching of a metal chelate probe, Eu³⁺-3,4,3-LI(1,2-HOPO), which only requires 10 min incubation before measurement. Gadolinium-based contrast agents prevent the formation of the Eu³⁺-3,4,3-LI(1,2-HOPO) complex, subsequently decreasing the luminescence of the assay solution. Three commercial contrast agents, Magnevist®, Multihance® and Omniscan®, are used to demonstrate the analytical concept in synthetic human urine, and subsequent quantification of mouse urine samples. This is believed to be the first assay capable of detecting and quantifying gadolinium-based contrast agents in urine without sample preparation or digestion.

Herein is reported a luminescence assay to detect and quantify GBCAs in urine. The emission quenching of the Eu³⁺-3,4,3-LI(1,2-HOPO) complex by Gd³⁺ is exploited through a competition reaction as analytical principle. The assay performance is initially demonstrated in a synthetic urine matrix, and three commercial GBCAs with linear structures (Magnevist®, Multihance® and Omniscan®) are used as analytes. The binding affinities between the contrast agent ligands and Gd³⁺ cations are found to determine the limit of detection and dynamic range for this analytical protocol. Finally, the assay performance is successfully tested in mouse urine samples.

### RESULTS AND DISCUSSION

Eu³⁺-3,4,3-LI(1,2-HOPO) as a luminescent bio-inspired probe for gadolinium detection

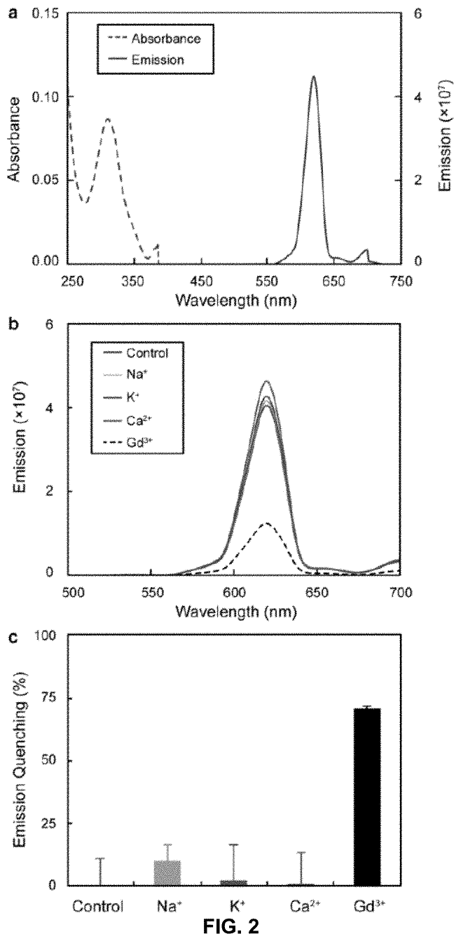

The spectroscopic properties of 20 μM Eu³⁺-3,4,3-LI(1,2-HOPO) are initially evaluated (FIG. 2a). A main absorption band at 315 nm is observed due to π-43 π* transitions that can be used to sensitize the emission of certain lanthanides.²⁴ The emission spectra under 315 nm excitation shows a sharp band centered at 620 nm, characteristic of Eu^{3+ 5}D_{0→}⁷F₂ hypersensitive transition.²⁴ The emission band is at a region of the visible spectrum where urine and fluorescent amino acids (e.g. tryptophan and tyrosine) are not optically active,^{30,31} minimizing matrix interferences. Hence, the intensity variation at 620 nm is further explored as analytical signal for the assay. The optical response (FIG. 2b) of the Eu³⁺-3,4,3-LI(1,2-HOPO) complex to Gd³⁺ ions and several potential interfering cations is examined. FIG. 2c shows a 71±1% quenching of 20 μM Eu³⁺-3,4,3-LI(1,2-HOPO) emission in the presence of 50 μM Gd³⁺, which is within the concentration range of GBCAs detected in urine of patients who have undergone Mill procedures.³² The luminescence decrease is caused by Gd³⁺ preventing Eu³⁺ complexation, due to 3,4,3-LI(1,2-HOPO) higher binding affinity for the former (log β₁₁₀ of 20.5 and 20.2 for Gd³⁺ and Eu³⁺, respectively²⁷). No significant luminescence variation is observed when Eu³⁺ and 3,4,3-LI(1,2-HOPO) are mixed with Na⁺, K⁺ and Ca²⁺ (the three most abundant cations found in urine³³), confirming the selectivity of the probe for Gd³⁺.

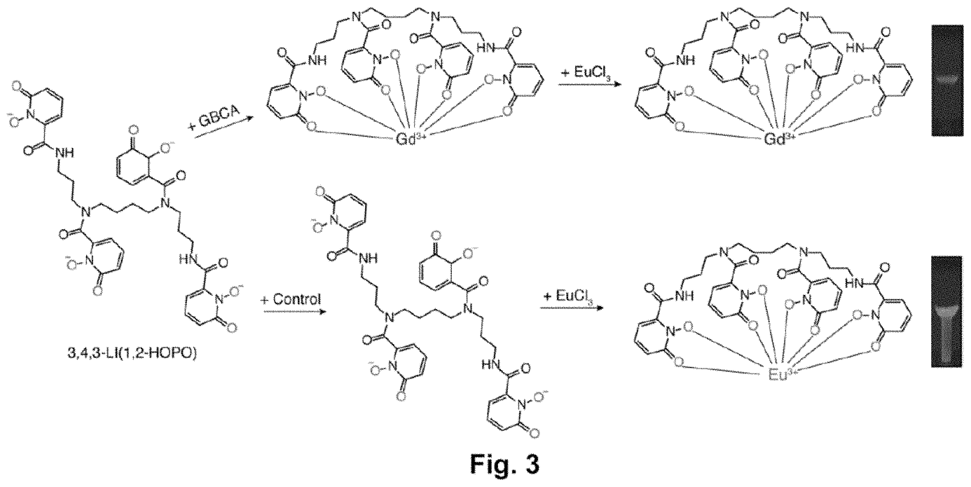

In light of these results, an analytical assay for GBCA detection based on the competition between Eu³⁺ and Gd³⁺ for 3,4,3-LI(1,2-HOPO) is designed. In this assay, 3,4,3-LI(1,2-HOPO) is added to the urine sample and, in the absence of GBCAs, 1,2-HOPO groups remain available to complex other metal species. Eu³⁺ is then added, and its emission is sensitized (FIG. 3). In the presence of GBCAs, however, 1,2-HOPO units acquire the Gd³⁺ from the contrast agent because of their higher affinity for the metal compared to clinical ligands,²³ preventing the coordination of subsequently added Eu³⁺. Thus, a drastic emission quenching is induced in presence of GBCAs.

In vitro sensing of GBCAs in urine

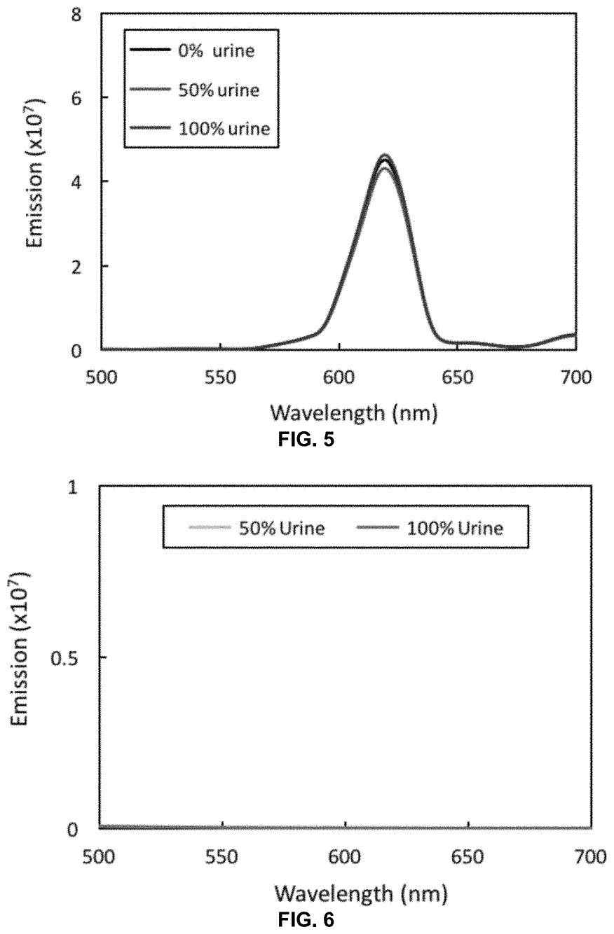

Urine is a complex matrix comprised of salts and metabolism by-products, which can potentially affect assay response. Thus, the effects of urine on the luminescence of the Eu³⁺-3,4,3-LI(1,2-HOPO) probe is analyzed using artificial human urine (Surine), which is often used for the optimization of analytical protocols.³⁴⁻³⁶ The emission of Eu³⁺-3,4,3-LI(1,2-HOPO) is not affected by urine, and the emission intensity is constant in different solutions ranging from 0 to 100% urine volume (FIG. 5). Although the average pH of human urine is 6.0, there is a strong variation among individuals.³⁷ Therefore, the following in vitro experiments are performed with urine at 50% dilution with IVIES buffer (50 mM, pH 6.0) to fix the acidity of the solution, preventing potential variations in the assay response due to sample pH, and 10 min incubation times, which had been optimized in preliminary experiments.

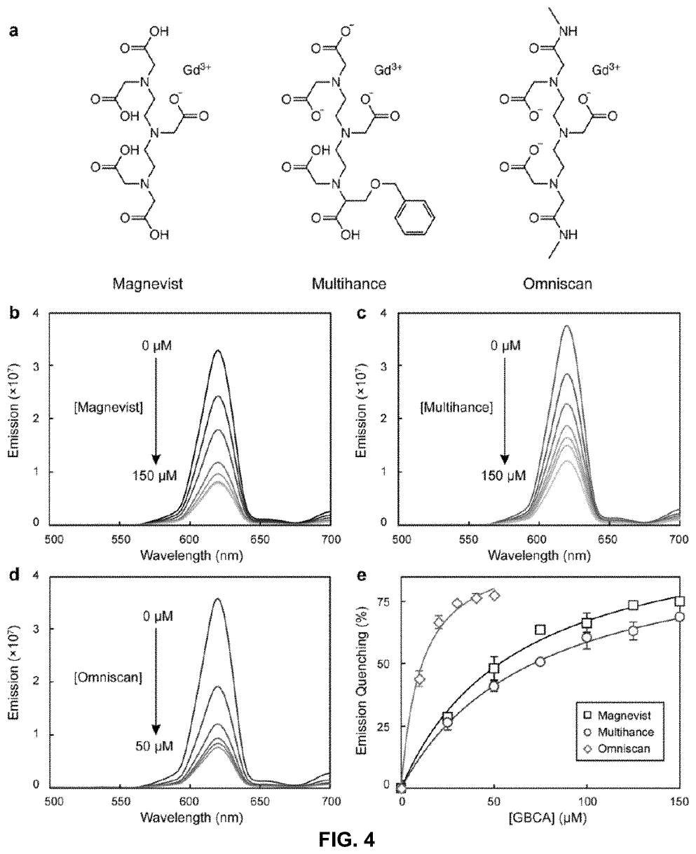

Given that linear GBCAs are the primary source of safety concerns, ¹⁵ three commercial contrast agents with linear ligands (FIG. 4a) are selected for the in vitro detection of GBCAs in urine experiments: Magnevist® (Gadopentetate), Multihance® (Gadobenic acid) and Omniscan® (Gadodiamide). The luminescence of the assay solution (20 μ³⁺ and 20 μM 3,4,3-LI(1,2-HOPO) in 50% synthetic human urine and 50 mM MES at pH 6.0) decreases by 75±1% as the concentration of Magnevist® increased up to 150 μM (FIG. 4b). Similar trends are observed with Multihance® and Omniscan® analytes (FIGS. 4c and 4d). The calibration curves of the assay are plotted as luminescence quenching vs GBCA concentration (FIG. 4e), and analytical parameters, such as the limit of detection and dynamic range, are described in Table 1. The response curves obtained for Magnevist® and Multihance® appeared similar, with both analytes reaching saturation at around 150 μM GBCA. This similarity is attributed to closely related chelators (DTPA and BOPTA, respectively), which display similar binding affinities for Gd³⁺.³⁸ Conversely, Omniscan® displayed a lower limit of detection (0.9 μM) and narrower dynamic range (from 0.9 to 50 μM), since its chelator has a lower affinity for the meta1.²³ Because the assay is based on the ability of 3,4,3-LI(1,2-HOPO) to acquire Gd' from a given GBCA, the binding affinity of the GBCA ligand for the Gd' affects the analytical response. Thus, calibration curves need to be performed using calibration standards for each GBCA analyzed, and one curve is not valid for all contrast agents.

TABLE 1
Summary of analytical response of the assay to GBCAs.

| | Limit of detection (μM) | Dynamic range (μM) |
|---|---|---|
| Magnevist | 6.1 | 6.1-150 |
| Multihance | 2.3 | 2.3-150 |
| Omnsican | 0.9 | 0.9-50 |

One concern for accurate GBCA quantification could be that the Gd³⁺ cation is released from the GBCAs, and complexed by other biomolecules, subsequently forming multiple metal complexes with different analytical responses. However, previous mass spectrometry analyses of linear GBCAs excreted through urine show that the complexes remain intact, and no free Gd³⁺ or other gadolinium-containing species can be detected.³⁹ Therefore, the trans-metalation of Gd³⁺ is not expected to be significant enough to affect the measurements in urine. The corrected limits of detection (accounting for the 50% urine sample dilution) are compared to reported levels of GBCAs in patient urine at different excretion time points (Table 2). For the three studied GBCAs, the limits of detection of the assay cover GBCA levels in patient urine up to 19 h post-injection (wherein 99.9% of excreted contrast agent has been eliminated by healthy patients³²), and up to 3 days after injection for Multihance® and Omniscan®.

TABLE 2
Comparison between assay response and reported
levels of GBCA in patient urine.

| | | [GBCA] (μM) |
|---|---|---|
| Adjusted limit of detection for | Magnevist | 12.2 |
| | Multihance | 4.6 |
| | Omnsican | 1.8 |

| | | | |
|---|---|---|---|
| GBCA in patient urine after | 3 | h³² | 7955 |
| injection | 6 | h³² | 1294 |
| | 12 | h³² | 550 |
| | 19 | h³² | 34.3 |
| | 3 | days⁴⁰ | 6.8 |

Quantification of GBCAs in mouse urine

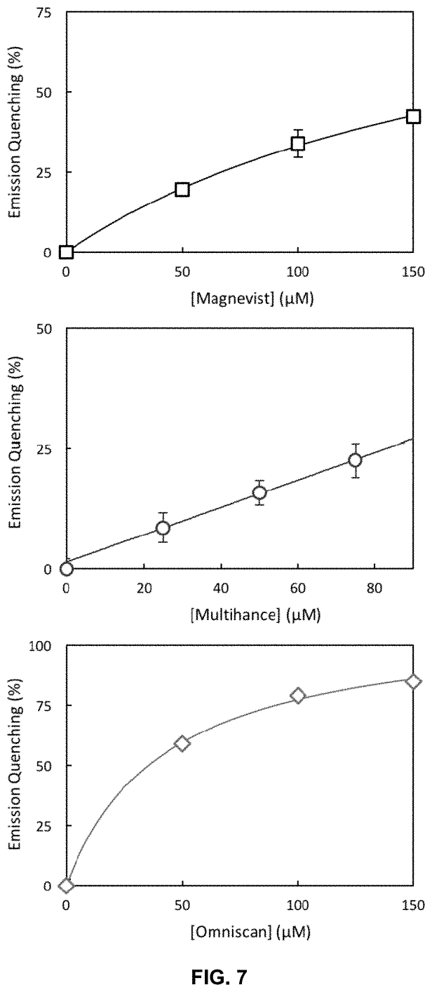

Finally, the luminescence assay by quantifying GBCAs in mouse urine samples is validated. The three GBCAs are intravenously injected (four mice per group), and urine is collected 2 h after injection, as mice excrete the contrast agents more quickly than humans do.⁴¹ Due to the different composition between mouse and human urine,⁴² calibration curves (FIG. 7) are performed by spiking GBCA standards into urine from control mice rather than into synthetic human urine. Table 4 reports the assay response to the mouse urine samples, and Table 3 compares the GBCA concentrations quantified by the assay versus inductively coupled plasma optical emission spectrometry (ICP-OES), which is one of the gold standard techniques for gadolinium quantification.⁴³ The assay provided values that are very similar to those of ICP-OES, with only slightly higher standard deviations. Furthermore, the assay does not require sample preparation (only 10 min incubation), while ICP-OES needs multi-step digestion that requires several hours before quantification as well as more expensive instrumentation.

TABLE 3
Quantification of GBCAs in mouse urine
samples collected 2 h after injection.

| | Luminescence assay | ICP-OES |
|---|---|---|
| [Magnevist] (M) | 0.020 ± 0.005 | 0.018 ± 0.000 |
| [Multihance] (M) | 0.017 ± 0.004 | 0.015 ± 0.000 |
| [Omnsican] (M) | 0.020 ± 0.001 | 0.024 ± 0.000 |

TABLE 4
Quantification of GBCAs in mouse urine
samples collected 2 h after injection.

| | Emission | [GBCA] in assay | [GBCA] in original |
|---|---|---|---|
| | Quenching (%)* | solution (μM) | sample (M) |
| Magnevist | 32.3 ± 3.0 | 101 ± 13 | 0.020 ± 0.005 |
| Multihance | 25.8 ± 2.7 | 86 ± 9 | 0.017 ± 0.004 |
| Omnsican | 77.8 ± 0.6 | 101 ± 3 | 0.020 ± 0.001 |

*Emission quenching at 620 nm under 315 nm excitation.

### Materials

3,4,3-LI(1,2-HOPO) is prepared and characterized as previously described (Abergel, R. J.; Durbin, P. W.; Kullgren, B.; Ebbe, S. N.; Xu, J.; Chang, P. Y.; Bunin, D. I.; Blakely, E. A.; Bjornstad, K. A.; Rosen, C. J.; Shuh, D. K.; Raymond, K. N. Biomimetic actinide chelators: an update on the preclinical development of the orally active hydroxypyridonate decorporation agents 3,4,3-LI(1,2-HOPO) and 5-LIO(Me-3,2-HOPO). Health Phys. 2010, 99 (3), 401-407). Surine negative urine control, europium (III) chloride hexahydrate 99.99%, gadolinium (III) chloride hexahydrate 99%, 2-(N-morpholino)ethanesulfonic acid (MES), nitric acid (70%) and sodium hydroxide 97% are purchased from Sigma-Aldrich (St. Louis, Mo.). Hydrogen chloride 0.1 N and 6 N is bought from VWR International (Radnor, Pa.). Magnevist® is purchased from Bayer (Leverkusen, Germany). Multihance® is bought from Bracco Diagnostics (Milan, Italy). Omniscan® is purchased from GE Healthcare (Chicago, Ill.). All solutions are prepared with Milli-Q water.

Photophysical Characterization of the Eu³⁺-3,4,3-LI(1,2-HOPO) complex

2 μL 3,4,3-LI(1,2-HOPO) (1 mM) and EuCl₃ (1 mM) are added to a water solution, mixed for 10 s, and left to incubate at room temperature, without stirring, for 10 min. The absorption spectrum (from 250 to 425 nm) and luminescence spectrum (from 500 to 750 nm under 315 nm excitation) are recorded with a SpectraMax iD3 Multi-Mode Microplate Reader (Molecular Devices, Sunnyvale, Calif.).

Selectivity of the Eu³⁺-3,4,3-LI(1,2-HOPO) probe

2 μL 3,4,3-LI(1,2-HOPO) (1 mM) are added to various solutions containing 50 μM of Gd³⁺, Na⁺, K⁺, Ca²⁺ or Mg²⁺, mixed for 10 s, and left to incubate at room temperature, without stirring for 10 min. 2 μL EuCl₃ (1 mM) are added to the solutions, mixed for 10 s, and left undisturbed for 10 min before recording emission spectra from 500 to 700 nm, under 315 nm excitation with a SpectraMax iD3 Multi-Mode Microplate Reader.

### Detection of GBCA in Synthetic Human Urine

Surine™ samples containing different amounts of GBCA are diluted down to 50% with MES solution (final concentration 50 mM, pH 6.0). 2 μL 3,4,3-LI(1,2-HOPO) (1 mM) are added to each solution, mixed for 10 s, and left to incubate at room temperature, without stirring for 10 min. 2 μL EuCl₃ (1 mM) are added, mixed for 10 s and left undisturbed for 10 min before recording emission spectra from 500 to 700 nm under 315 nm excitation with a SpectraMax iD3 Multi-Mode Microplate Reader.

The limit of detection is calculated as:

I_{LOD}=I_{blank}+3·σ_{blank}

Where I_{LOD} is the assay response intensity at the limit of detection, I_{blank} is the response intensity of the blank, and σ_{blank} is the standard deviation for the blank signal.

To quantify the concentrations of the GBCA stock solutions, 50 μL of commercial stock solutions are digested in 100 μL of nitric acid (70%) and 100 μL of hydrogen chloride (6 N) for 60 min at room temperature and re-dispersed in 4.75 mL of Milli-Q water, following a previously published protocol (Pallares, R. M.; Thanh, N. T. K.; Su, X. Tunable plasmonic colorimetric assay with inverse sensitivity for extracellular DNA quantification. Chem. Commun. 2018, 54 (80), 11260-11263). The final concentration of gadolinium in solution is quantified by Inductively Coupled Plasma Optical Emission Spectroscopy (ICP-OES, Perkin Elmer, Waltham, Mass.).

### Animal Procedure

The protocol used in the animal studies is reviewed and approved by the Institutional Animal Care and Use Committee of Lawrence Berkeley National Laboratory, and is performed in AAALAC-accredited facilities according to prescribed guidelines and regulations. The animals are young adult (78 days old) female (33.0±1.4 g) Swiss Webster mice (Simonsen Laboratories, Gilroy, Calif., USA). Mice are kept under a 12 h light cycle with controlled temperature (21-24° C.) and relative humidity (30-70%), and are given water and food ad libitum. Each group is housed together in a plastic stock cage lined with a 0.5 cm layer of highly absorbent low-ash pelleted cellulose bedding (ALPHA-dri). Groups of four mice are injected intravenously into a warmed lateral tail vein with a single dose of GBCA (0.3 mmol/kg mouse) or bacteriostatic 0.9 bio sodium chloride solution (control group), and urine is collected after 2 h through bladder massage method. Finally, euthanasia is performed under isotiurane anesthesia.

### Detection of GBCA in Mouse Urine

Because the GBCA concentration in mouse urine is very high at early excretion times, the samples are diluted in buffer as described: 1 μL mouse urine samples are mixed with 191 MES solutions (final concentration 50 mM, pH 6.0). 4 μL 3,4,3-LI(1,2-HOPO) (1 mM) are added to each solution, mixed for 10 s, and left to incubate at room temperature, without stirring for 10 min. 4 μL EuCl₃ (1 mM) are added, mixed for 10 s and left undisturbed for 10 min before recording emission spectra from 500 to 700 nm under 315 nm excitation with a SpectraMax iD3 Multi-Mode Microplate Reader. For calibration curve preparation, GBCA standard solutions are spiked into urine of control mice. The resulting solutions are quantified as previously described in this paragraph.

To quantify the concentrations of the GBCA in mouse urine by ICP-OES, 50 μL of commercial stock solutions are digested in 100 μL of nitric acid (70%) and 100 μL of hydrogen chloride (6 N) for 3 h at 60 ° C. and re-dispersed in 4.75 mL of Milli-Q water. The final concentration of gadolinium in solution is quantified on a Perkin Elmer 5300 DV optical emission ICP with auto sampler at the Inductively Coupled Plasma Spectroscopy Facility of the University of California, Berkeley. Gd detection limit for this instrument is 0.9 μg/L.

### Conclusions

In summary, a luminescence assay is developed for the detection of GBCAs in urine based on competition between Eu³⁺ and Gd³⁺ for 3,4,3-LI(1,2-HOPO). In the absence of a contrast agent, the chelator complexes Eu' and sensitizes its emission. If the urine sample contains a GBCA, however, 3,4,3-LI(1,2-HOPO) acquires the Gd³⁺ from the contrast agent, preventing Eu³⁺ coordination and quenching its luminescence. Because 3,4,3-LI(1,2-HOPO) is stable in urine and has a higher binding affinity for Gd' than clinical ligands do, the assay does not require sample digestion and can be performed directly in urine. Given that the assay requires minimal equipment and provides faster results than gold standard techniques do (10 min compared to several hours), it is expected this versatile quantitative method will improve accessibility to GBCA quantification and optimize patient follow-ups after MM procedures.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to detect and/or quantify a gadolinium-based contrast agents (GBCA) in a sample, the method comprising: (a) providing a sample, (b) contacting the sample with a luminescent agent so that luminescent agent chelates a Gd³⁺ cation of the GBCA, and (c) measuring quenching of a luminescence emission within a range of wavelength; wherein the quenching of the luminescence emission corresponds to the amount of luminescent agent chelating a Gd³⁺ cation.

2. The method of claim 1, wherein the providing step (a) comprises obtaining the sample from a subject.

3. The method of claim 1, wherein the sample obtained from the subject is not further treated, prepared, or digested prior to the contacting step (b).

4. The method of claim 1, wherein the contacting step (b) comprises incubating the sample and the luminescent agent together.

5. The method of claim 1, wherein the measuring step (c) comprises: (i) exposing the luminescent agent to one or more, or a range of, wavelengths of light to cause the luminescent agent to produce the luminescence emission, (ii) measuring the amount of luminescence emission, and (iii) comparing the amount of luminescence emission measured in (ii) to an amount of luminescence emission of a luminescent agent mixed with a blank, negative sample control, or not contacted to the sample.

6. The method of claim 1, wherein the luminescent agent in contacting step (b) is chelated to Eu³⁺.

7. The method of claim 1, wherein the range of wavelength is from about 590 nm to 640 nm.

8. The method of claim 7, wherein the range of wavelength is about 620 nm.

9. The method of claim 1, wherein the gadolinium-based contrast agent (GBCA) is gadopentetate, gadobenate, or gadodiamide.

10. The method of claim 1, wherein the sample is a bodily fluid or a fluid obtained from a body.

11. The method of claim 10, wherein the sample is blood, serum, or urine.

12. The method of claim 1, wherein the luminescent agent is a 1,2-HOPO chelating agent or 3,2-HOPO chelating agent.

13. The method of claim 12, wherein the luminescent agent is a 1,2-HOPO chelating agent defined by the structure: 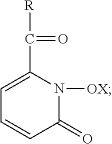
wherein R is a hydroxy group or 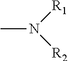
where R₁ and R₂ are selected from the group consisting of H, —CH₃, —CH₂CH₃ and CH₂-φ, and X is either hydrogen, an alkali metal ion, or a quaternary ammonium ion.

14. The method of claim 13, wherein the 1,2-HOPO chelating agent comprises a following structure: 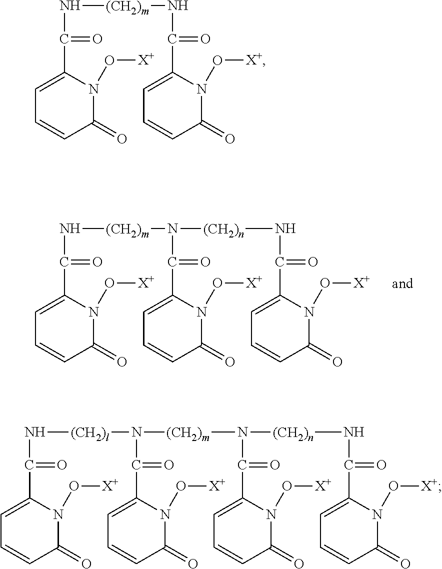
wherein l, m and n are integers between one and twenty.

15. The method of claim 12, wherein the luminescent agent is a 3,2-HOPO chelating agent defined by the structure: 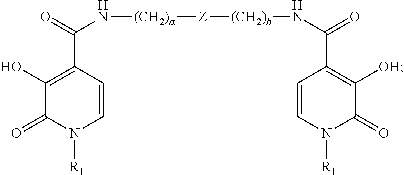
wherein R₁ is a member selected from the group consisting of hydrogen, C₁₋₄ aliphatic hydrocarbon groups, and C₁₋₄ aliphatic hydrocarbon groups substituted by a single halide, hydroxy, carboxy, or aryl group; Z is a member selected from the group consisting of O, NH, N-alkyl, and N-aryl; a is 2-4; and b is 2-4.
